(19) United States
(12) Patent Application Publication
(10) Pub. No.: US 2007/0098789 A1
(43) Pub. Date: May 3, 2007

(54) Organoleptically acceptable ibuprofen oral dosage formulations, methods of making and using the same

(76) Inventor: Toru Hibi, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/582,850

(22) Filed: Oct. 17, 2006

Related U.S. Application Data
(60) Provisional application No. 60/733,127, filed on Nov. 2, 2005.
(60) Provisional application No. 60/810,417, filed on Jun. 1, 2006.

(51) Int. Cl. A61K 31/724; A61K 31/192; A61K 36/534; A61K 9/20

(52) U.S. Cl. 424/464; 514/58; 514/569; 424/747

(57) ABSTRACT

Organoleptically acceptable solid oral dosage formulations of ibuprofen, and methods of making and using the same, are provided. A feature of the subject formulations is that they include ibuprofen and a masking component. In certain embodiments, the masking component includes one or more of a cooling agent, an organic acid and a cyclodextrin. The subject invention finds use in a variety of applications.

### CROSS-REFERENCE TO RELATED APPLICATIONS

Pursuant to 35 U.S.C. § 119 (e), this application claims priority to the filing dates of: U.S. Provisional Patent Application Ser. No. 60/733,127 filed Nov. 2, 2005 and U.S. Provisional Patent Application Ser. No. 60/810,417 filed on Jun. 1, 2006; the disclosures of which are herein incorporated by reference.

### INTRODUCTION

### BACKGROUND OF THE INVENTION

Sore throat, laryngitis, mouth and throat ulcers, excessive mucus and other mouth and throat irritations typically accompany common colds, influenza and like ailments. A variety of different medications, including medications available over-the-counter, have been developed to treat these types of maladies. Such medications include: Dyclonine lozenges (e.g., as sold under the trademark SUCRETS™); benzocaine+menthol lozenges (e.g., as sold under the trademark CEPACOL™); menthol lozenges (e.g., as sold under the trademark VICKS™); and aspirin containing chewing gum (e.g., as sold under the trademark ASPERGUM™).

Ibuprofen (2-(p-isobutylphenyl)propionic acid) is a non-steroidal anti-inflammatory agent (NSAID) which is known to possess analgesic and antipyretic activities. It is useful in the treatment of pain and inflammation associated with various maladies, including the common cold, toothaches, headaches, backaches, menstrual cramps (Dysmennorhea), the muscular aches and pains associated with Premenstrual Syndrome, rheumatoid arthritis and osteoarthritis, as well as in the reduction of fever.

As such, like other NSAIDs, ibuprofen has become widely used in prescription and over-the-counter formulations for the treatment of pain associated with inflammation, both minor and chronic. One of its drawbacks, however, is that Ibuprofen has an unpleasant, bitter taste which tends to limit its acceptability in many oral dosage forms. Methods of alleviating this limitation have included attempts at masking the bitter taste with flavored and/or sweetened mediums or by coating the ibuprofen with substances which prevent it from contacting the taste buds during oral administration. For example, oral ibuprofen formulations currently available over the counter include suspensions of ibuprofen in oral sugar syrup containing formulations.

JP4-26618 describes ibuprofen containing lozenges and their use in the treatment of sore throat. To overcome the bitterness of the ibuprofen active agent and thereby make the lozenge organoleptically acceptable, the disclosed lozenge formulations include a significant amount of cyclodextrin (e.g., at least twice as much cyclodextrin as ibuprofen) as a masking agent.

Because of the relatively expensive cost of cyclodextrin, there is continued interest in the development of new organoleptically acceptable oral ibuprofen formulations, e.g., formulations in which cyclodextrin is present in smaller amounts than that taught in JP-4-26618, if at all.

Relevant Literature

U.S. Pat. Nos. 5,024,997; 5,055,461; 5,780,046; 6,166,083; 6,194,003; 6,517,870; and 6,616,083. Also of interest are Japanese Patent Publication Nos. Sho.62-298528 and Hei.4-26618. Other references of interest include: Breslin et al., Chem. Senses (2001) 26:55-65; Hahn, Int.J.Clin.Pharm.Res. VI(1) 81-86(1986); Schactel et al., Clin. Pharmacol. Ther. (1988) 44:704-711; and Wilson et al., Drugs made in Germany 38, No.3(1995).

### SUMMARY OF THE INVENTION

Organoleptically acceptable solid oral dosage formulations of ibuprofen, and methods of making and using the same, are provided. A feature of the subject formulations is that they include ibuprofen and a masking component. In certain embodiments, the masking component includes one or more of a cooling agent, an organic acid and a cyclodextrin. The subject invention finds use in a variety of applications.

### DETAILED DESCRIPTION

Organoleptically acceptable solid oral dosage formulations of ibuprofen, and methods of making and using the same, are provided. A feature of the subject formulations is that they include ibuprofen and a masking component. In certain embodiments, the masking component includes one or more of a cooling agent, an organic acid and a cyclodextrin. The subject invention finds use in a variety of applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As reviewed above, the present invention provides organoleptically acceptable ibuprofen oral solid dosage formulations, as well as methods for making and using the same. In further describing representative embodiments of the invention in greater detail, the organoleptically acceptable formulations are reviewed first in greater detail, followed by a review of representative protocols for making the formulations and a review of representative applications in which the formulations find use.

Organoleptically Acceptable Ibuprofen Oral Solid Dosage Formulations

As summarized above, the subject invention provides organoleptically acceptable ibuprofen oral solid dosage formulations. As the formulations are organoleptically acceptable, they can contact the taste receptors of a recipient's mouth and be considered generally acceptable to the senses of the recipient, particularly to the sense of taste. More particularly, the organoleptically acceptable formulations of this invention are those solid oral formulations in which the unpleasant and bitter taste of ibuprofen is sufficiently masked. Specifically, when using the evaluation protocol reported in the Experimental Section below, the unpleasant and bitter taste of ibuprofen is considered to be sufficiently masked if the composition scores a 1 or less, e.g., 0 or less, such as −1 or less, including −2.

In a general sense, the subject formulations are not limited to ibuprofen formulations, but instead may be viewed as propionic acid derivative formulations. Propionic acid derivatives are a well known class of analgesic compounds. As used herein propionic acid derivatives are understood to include, but are not limited to, ibuprofen, naproxen, benoxaprofen, naproxen sodium, flurbiprofen, fenoprofen, fenbuprofen, ketoprofen, indoprofen, pirprofen, carpofen, oxaprofen, pranoprofen, microprofen, tioxaprofen, suproprofen, alminoprofen, tiaprofenic acid, fluprofen and bucloxic acid. The structural formula is set forth in U.S. Pat. No. 4,923,898, hereby incorporated by reference. Propionic acid derivatives as defined herein are defined as pharmaceutically acceptable analgesics/non-steroidal anti-inflammatory drugs having a free —CH(CH₃)COOH or —CH₂ CH₂ COOH or a pharmaceutically acceptable salt group, such as —CH(CH₃)COO—Na⁺ or CH₂ CH₂ COO—Na⁺, which are typically attached directly or via a carbonyl functionality to an aromatic ring system.

For convenience and ease of description, the present invention is described herein, primarily in terms of ibuprofen formulation embodiments. As such, the subject formulations of these representative ibuprofen formulation embodiments include an effective amount of ibuprofen. Ibuprofen is a widely used, well known non-steroidal anti-inflammatory propionic acid derivative. Ibuprofen is chemically known as 2-(4-isobutylphenyl)-propionic acid. As used herein, ibuprofen is understood to include 2-(4-isobutylphenyl)propionic acid as well as pharmaceutically acceptable salts thereof. Suitable ibuprofen salts include arginine, lysine, histidine, as well as other salts described in U.S. Pat. No. 4,279,926, 4,873,231, 5,424,075 and 5,510,385, the contents of which are incorporated by reference. It should be noted that the ibuprofen active agent may be present as a racemic mixture or as a stereoisomer, e.g., as the S(+) or R(−) ibuprofen sterioisomers.

The amount of ibuprofren present in the subject formulations may vary, so long as it is effective to acheive the intended purpose of the formulation, e.g., to provide sore throat pain relief to a subject in need thereof, as further reviewed below. In representative embodiments, the amount of ibuprofen present in the formulation ranges from about 5 to about 600 mg, such as from about 20 to about 400 mg, including from about 50 to about 200 mg.

In addition to the ibuprofen active agent, the subject formulations also include a masking component. By masking component is meant a component that is made up of one or more agents which provides for sufficient masking of the ibuprofen bitterness to make the formulation organoleptically acceptable. In representative embodiments, the masking component is made of one or or more of a cooling agent, an organic acid and a cycodextrin. In certain embodiments, the masking component includes two or more of a cooling agent, an organic acid and a cyclodextrin, including all three of a cooling agent, an organic acid and a cyclodextrin.

As such, certain embodiments of the subject invention include one or more cooling agents. By “cooling agent” is meant an agent that, when contacted with skin of a subject, imparts a cooling sensation or effect to the subject. Cooling agents can be selected from any of a wide variety of materials. Included among such materials are carboxamides, menthol, ketals, diols, and mixtures thereof. In certain embodiments, the cooling agent is an acyclic amide, where representative acyclic amides include compounds of the formula: 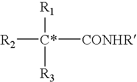
where

R₁, R₂ and R₃ are each C₁-C₅ alkyl and together provide a total of at least 5, such as from about 5-10 carbon atoms; and R′ is C₁-C₅ alkyl, C₁-C8 hydroxyalkyl or alkylcarboxyalkyl of up to 8 carbon atoms. In this group R₁ is in representative embodiments, methyl, ethyl or n-propyl and one or both of R₂ and R₃ is branched in an alpha or beta position relative to the carbon atom marked (*). In representative embodiments, the cooling agent is N,2,3-trimethyl-2-isopropyl butamide (also known as WS-23; CAS # 51115-67-4). The above compounds can be produced using any convenient protocol, where representative protocols are described in U.S. Pat. No. 4,296,255. Other representative cooling agents of interest include, but are not limited to: linalool, geraniol, hydroxycitronellal, cyclohexanecarboxamide, N-ethyl-5-metyhy-2-(1-methylethyl) (also known as WS-3; CAS # (39711-79-0), Flescolat MGA (Haarman & Reimer), Frescolat M L (Haarmann & Reimer), PMD38 (Takasago), CoolactP (Takasago) and Cooling Agent 10 (Takasago). Additional preferred cooling agents are selected from the group consisting of menthol, 3-1-menthoxypropane-1,2-diol known as TK-10 manufactured by Takasago; menthols and menthyls, where these as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosures of which are incorporated herein by reference, as well as various oils, such as peppermint oil, spearmint oil, and the like.

The amount of cooling agent that is present in the formulation is an amount sufficient (e.g., by itself or in combination with other masking agents of the masking component) to mask or hide the bitterness of ibuprofen and thereby make the formulation organoleptically acceptable. In representative embodiments, based on the ratio to ibuprofen, the amount ratio of cooling agent to ibuprofen present in the formulation ranges from about 0.25 to about 2,such as from about 0.5 to about 1.5,and including from about 0.5 to about 1.

The masking component may also include one or more organic acids, including amino acids. Organic acids of interest include, but are not limited to: glycolic acid, lactic acid, methyl lactic acid, palycarobxlyic acids, e.g., malic acid, citric acid, tartronic acid, tartaric acid, succinic acidetc. Amino acids of interest include, but are not limited to: glycine, alanine, valine, leucine, isoleucine, serine, threonine, cysteine, cystine, methionine, aspartic acid, asparagine, glutamic acid, glutamine, arginine, lysine, 5-hydroxylysine, histidine, phenylalanine, tyrosine, tryptophan, 3-hydroxyproline, 4-hydroxyproline, proline, homocysteine, homocystine, homoserine, ornithine, citrulline, creatine, asparaginic acid, 3-aminopropanoic acid, theanine, 2-aminobutanoic acid, 4-aminobutanoic acid, 2-amino-2-methylpropanoic acid, 2-methyl-3-aminopropanoic acid, 2,6-diaminopimelic acid, 2-amino-3-phenylbutanoic acid, phenylglycine, canavanine, canaline, 4-hydroxyarginine, 4-hydroxyornithine, homoarginine, 4-hydroxyhomoarginine, β-lysine, 2,4-diaminobutanoic acid, 2,3-diaminopropanoic acid, 2-methylserine, 3-phenylserine betaine, sulfur-containing amino acids, such as taurine, cysteinesulfinic acid, methionine sulfoxide and methionine sulfone.

The amount of organic acid (including amino acid) masking agent that is present in the formulation is an amount sufficient (e.g., by itself or in combination with other masking agents of the masking component) to mask or hide the bitterness of ibuprofen and thereby make the formulation organoleptically acceptable. In representative embodiments, based on the ratio to ibuprofen, the amount ratio of organic acid to ibuprofen present in the formulation ranges from about 0.5 to about 4, such as from about 1 to about 3, and including from about 1 to about 2.

In representative embodiments, the masking component includes a cyclodextrin. The cyclodextrin may be any convenient cyclodextrin or mixture of cyclodextrins, including α-, β- or γ-cyclodextrins. In representative embodiments, cyclodextrin is β-cyclodextrin. A feature of embodiments of the invention is that, when present, the total amount of cyclodextrin in a given formulation is less than twice the amount of ibuprofen active agent in the formulation, such as less than about 1.5 times the amount of ibuprofen active agent, including less than about 1 times the amount of ibuprofen active agent, in terms of mass.

As summarized above, the subject formulations are orally acceptable solid formulations. The solid formulations may be present in a number of different formats, where representative formats include, but are not limited to: lozenges, troches, tablets, liquid formulations such as gargles and sprays, and gums. The term “lozenge” as used herein is intended to embrace all dosage forms where the product is formed by cooling a sugar-based or sugar alcohol based (e.g., sorbitol) molten mass containing the active material. The term “tablet” as used herein is intended to embrace unit dosage forms made from compressed powders or granules or compressed pastes. Solid dosage forms may be prepared by methods which are well known in the art for the production of lozenges, tablets, troches, capsules or chewing gums and may contain other ingredients known in such dosage forms such as acidity regulators, opacifiers, stabilizing agents, buffering agents, flavorings, sweeteners, coloring agents, buffering agents, sweeteners and preservatives.

For example, solid formulations of the present invention may be prepared as lozenges by heating the lozenge base (e.g., a mixture of sugar and liquid glucose) under a vacuum to remove excess water. The remaining components are then blended into the mixture. The resulting mixture is then drawn into a continuous cylindrical mass from which the individual lozenges are formed. The lozenges are then cooled, subjected to a visual check and packed into suitable packaging. One form of suitable packaging is a blister pack of a water-impermeable plastics material (e.g., polyvinylchloride) closed by a metallic e.g., aluminium foil. The patient removes the lozenge by applying pressure to the blister to force the lozenge to rupture and pass through the metal foil seal. Lozenges will normally be sucked by the patient to release the ibuprofen. Where desired, ethanol can be used to dissolve ibuprofen, menthol, WS-23, and WS-3.

As for preparation of a troche, a wet granulation method can be used generally to prepare granules for tabletting into a troche formulation. Ethanol can be used to dissolve ibuprofen, menthol, WS-23, and WS-3, if necessary. After granulation, the wet granule is dried, then mixed with lubricant, and finally tabletted into a troche.

Masticable solid dosage formulations may be made by the methods used to prepare chewable candy products or chewing gums. For example, a chewable solid dosage form may be prepared from an extruded mixture of sugar syrup to which the ibuprofen has been added with optional addition of whipping agents, humectants, lubricants, flavors and colorings. (See Pharmaceutical Dosage Forms: Tablets, Volume 1, Second Edition edited by H A Lieberman, L Lachman and J B Schwartz published in 1989).

As such, a variety of different solid dosage formulations are provided by the subject invention. Furthermore, the solid dosage formulations do not need a special procedure for their preparation, as they may be readily produced using conventional procedures. For example, taste-masking agents, diluents, binders, or other appropriate additives can be added to ibuprofen, to which water or organic solvents are added, if necessary, and then mixed evenly to be compacted or to be granulated, and then mixed with lubricant to be compacted. For a diluent, sugar is mainly used and one or more types of sugar such as white sugar, powder sugar, lactose, fructose, starch syrup, reduced malt sugar, D- mannitol, D-sorbitol, and sucrose. For a binder, polyvinyl pyrrolidone, hydroxypropylcellulose, hydroxypropylmethylcellulose, corn starch, gelatin and arabic gum are used. For a lubricant, magnesium stearate, talc, sucrose fatty acid ester and such are properly selected and used.

In many embodiments, the methods of manufacture may be characterized by including a first step of producing an intermediate composition, which composition includes the active agent and masking component, and then a second step of producing the oral dosage formulation from the intermediate composition.

Methods of Using Subject Formulations

The subject organoleptically acceptable ibuprofen solid oral dosage formulations find use in applications of delivering ibuprofen to a subject in need thereof, particularly to a laryngopharynx (e.g., throat) location of a subject. In practicing the invention, the dosage may be placed in the mouth of the subject, e.g., by the subject itself or a caregiver therefore, whereupon the subject holds the formulation in its mouth to obtain the desired benefit, where the term holding is used broadly to include sucking, chewing, etc, depending on the particular type of formulation. In this manner, the direct action of ibuprofen dissolved in saliva or oral cavity is exerted on mucosal membrane, e.g., for treatment of sore throat.

In practicing the subject methods, a formulation may be administered a single time or a plurality of times over a given time period, e.g., the course of the disease condition, e.g., inflammation, being treated, where the dosing schedule when a plurality of formulations are administered over a given time period may be hourly, daily etc.

The above described formulations and methods find use in any application in which the administration of ibuprofen to a subject, particularly to a laryngopharynx location thereof, is desired. Among other applications, the subject methods as described herein are effective for treating inflammation, aches, etc., including the maladies reviewed in the introduction section of this application, e.g., sore throat, hoarse voice, etc.

Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In representative embodiments, the subject methods find use in the treatment of a sore throat. In yet other embodiments, the subject methods find use in the treatment of hoarse voice, e.g., as may occur from extended periods of voice use, e,g., speaking, singing etc. By treatment is meant at least an amelioration of pain afflicting the host, where amelioration is used in a broad sense to refer to at least a reduction in the magnitude of pain. As such, treatment also includes situations where the pain is completely inhibited, e.g. prevented from happening, or stopped, e.g. terminated, such that the host no longer suffers from the pain. As such, treatment includes both curing and managing a pain, e.g., of a sore throat.

Kits

Also provided are kits, where the subject kits at least include one or more, e.g., a plurality of, organoleptically acceptable oral solid dosage formulations, as described above. The subject formulations in the kits may be present in a package. The formulations of the kits are typically present in individual pouches or analogous containers, to preserve the composition of the formulations until use. The subject kits also generally include instructions for how to use the formulations, where the instructions typically include information about how to administer the formulation, dosing schedules etc. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following practical and comparative examples are offered by way of illustration and not by way of limitation.

### EXAMPLES

### 1. Working Example 1

After 1.0 g of ibuprofen and 0.5 g of I-menthol were dissolved in 2 ml of EtOH, the resultant solution was added to 4 g of D-sorbitol in a motor, and the resultant composition was kneaded well. After the composition was dried at room temperature overnight, it was pulverized by motor, sieved with 0.5 mm opening, and then mixed with 0.1 g of Mg-st (magnesium stearate) for 10 seconds. The prepared granules were put into a molder for tabletting (13 mm in diameter, in 1.5-2.0 t pressure for 20 secs.),to obtain 20 troches, each of which weighed 200 mg (36.4 mg of ibuprofen, 18.2 mg of I-menthol).

### 2. Working Example 2

1.0 g of ibuprofen and 0.5 g of WS-23 (commercialized cooling compound; N,2,3-trimethyl-2-isopropyl butamide; from Millennium Specialty Chemicals) were dissolved in 2 ml of EtOH, and the resultant solution was added to 4 g of D-sorbitol in a motor. The resultant composition was then kneaded well. Afterwards, the composition was dried at room temperature overnight, pulverized by motor and sieved with 0.5 mm opening. The resultant particles were then mixed with 0.1 g of Mg-st(magnesium stearate) for 10 sec. The prepared granules were then put into a molder for tabletting (13 mm in diameter, in 1.5-2.0 t pressure for 20 secs.) to obtain 20 troches, each of which weighed 200 mg (36.4 mg of ibuprofen, 18.2 mg of WS-23).

### 3. Working Example 3

1.0 g of ibuprofen and 0.5 g of WS-3(commercialized cooling compound; chclohexanecarboxamide, N-ethyl-5-methyl-2-(1-methylethyl); from Millennium Specialty Chemicals) were dissolved in 2 ml of EtOH, and the resultant solution was added to 4 g of D-sorbitol in a motor. The resultant composition was then kneaded well. Afterwards, the composition was dried at room temperature overnight, pulverized by motor and sieved with 0.5 mm opening. The resultant particles were then mixed with 0.1 g of Mg-st(magnesium stearate) for 10 seconds. The prepared granules were then put into a molder for tabletting (13 mm in diameter, in 1.5-2.0 t pressure for 20 secs.), to obtain 20 troches, each of which weighed 200 mg (36.4 mg of ibuprofen, 18.2 mg of WS-3).

### 4. Working Example 4

1.0 g of ibuprofen was dissolved in 2 ml of EtOH and 1.0 g of citric acid was dissolved in 1 ml of purified water, respectively. Both solutions were combined and mixed thoroughly using a vortex mixer. The resultant solution was added to 4 g of D-sorbitol in a motor. The resulting composition was then kneaded well. Afterwards, the composition was then dried at 50° C. under reduced pressure for 5 days. The composition was pulverized by a motor, sieved with 0.5 mm opening, then mixed with 0.1 g of Mg-st(magnesium stearate) for 10 seconds. The prepared granules were then put into a molder for tabletting (13 mm in diameter, in 1.5-2.0 t pressure for 20 secs.),to obtain 25 troches, each of which weighed 200 mg (33.3 mg of ibuprofen, 33.3 mg of citric acid).

### 5. Working Example5

1.0 g of ibuprofen was dissolved in 2 ml of EtOH and 1.0 g of malic acid was dissolved in 1 ml of purified water, respectively. Both solutions were combined and mixed thoroughly using a vortex mixer. The resultant solution was added to 4 g of D-sorbitol in a motor. The resultant composition was then kneaded well. Afterwards, the composition was dried at 50° C. under reduced pressure for 7 days, pulverized by a motor, sieved with 0.5 mm opening, then mixed with 0.1 g of Mg-st (magnesium stearate) for 10 seconds. The prepared granules were put into a molder for tabletting (13 mm in diameter, in 1.5-2.0 t pressure for 20 secs.)to obtain 25 troches, each of which weighed 200 mg (33.3 mg of ibuprofen, 33.3 mg of malic acid).

### 6. Working Example6

1.0 g of ibuprofen was dissolved in 2 ml of EtOH and 1.0 g of glutamic acid was dissolved in 1 ml of purified water, respectively. Both solutions were combined and mixed thoroughly using a vortex mixer. The resultant solution was added to 4 g of D-sorbitol in a motor. The resultant composition was then kneaded well. Afterwards, the composition was dried at room temperature overnight, pulverized by motor, sieved with 0.5 mm opening, then mixed with 0.1 g of Mg-st(magnesium stearate) for 10 seconds. The prepared granules were then put into a molder for tabletting (13 mm in diameter, in 1.5-2.0 t pressure for 20 secs.) to obtain 25 troches, ach of which weighed 200 mg (33.3 mg of ibuprofen, 33.3 mg of glutamic acid).

### 7. Working Example7

1.0 g of ibuprofen was dissolved in 2 ml of EtOH and 1.0 g of taurine was dissolved in 1 ml of purified water, respectively. Both solutions were combined and mixed thoroughly using a vortex mixer. The resultant solution was added to 4 g of D-sorbitol in a motor. The resultant composition was then kneaded well. Afterwards, the composition was dried at room temperature overnight, pulverized by motor, sieved with 0.5 mm opening, then mixed with 0.1 g of Mg-st(magnesium stearate) for 10 seconds. The prepared granules were put into a molder for tabletting (13 mm in diameter, in 1.5-2.0 t pressure for 20 secs.), to obtain 25 troches, each of which weighed 200 mg (33.3 mg of ibuprofen, 33.3 mg of taurine).

### 8. Working Example8

3.0 g of ibuprofen was dissolved in 4 ml of EtOH, and 3.0 g of beta-cyclodextrin was kneaded with 2 ml of purified water in motor, respectively. The ibuprofen solution was added to the component of beta-cyclodextrin in a motor. The resultant composition was then kneaded well. Afterwards, the composition was dried at room temperature overnight, pulverized by motor, sieved with 0.5 mm opening, then mixed with 0.1 g of Mg-st(magnesium stearate) for 10 seconds. The prepared granules were put into a molder for tabletting (13 mm in diameter, in 1.5-2.0 t pressure for 20 secs.), to obtain 60 troches, each of which weighed 80 mg (40 mg of ibuprofen, 40 mg of beta-cyclodextrin).

### 9. Working Example9 (as Comparative Sample)

1.0 g of ibuprofen was dissolved in 2 ml of EtOH and then 1 ml of purified water was added. The resultant solution was added to 4 g of D-sorbitol in a motor. The resultant composition was then kneaded well. Afterwards, the composition was dried at 50-60° C. overnight, pulverized by motor, sieved with 0.5 mm opening, then mixed with 0.1 g of talc for 10 seconds. The prepared granules were put into a molder for tabletting (13 mm in diameter, in 1.5-2.0 t pressure for 20 secs.),to obtain 20 troches, each of which weighed 200 mg (40 mg of ibuprofen).

### 10. Test Example

### Effectiveness to Reduce the Unacceptably Irritant Sensation of Ibuprofen

(Method) The unpleasant irritant sensation of ibuprofen is tested with panelists using the ibuprofen troche prepared in the procedures of working example 1-9 as a comparison sample.

(Result) As shown in Table 1, it is confirmed that the unpleasant irritant sensation of ibuprofen is reduced by adding the following ingredients, I-menthol, WS-23, WS-3, citric acid, malic acid, glutamic acid, taurine and beta-cyclodextrin.

TABLE 1

| Ingredient/working | Degree of Irritation* | | | | |
|---|---|---|---|---|---|
| example No. | −2 | −1 | 0 | +1 | +2 |
| l-Menthol/1 | | 1 | 1 | | |
| WS-23/2 | | 3 | | | |
| WS-3/3 | | | 2 | | |
| Citric acid/4 | | 1 | 1 | | |
| Malic acid/5 | | 1 | 1 | | |
| Glutamic acid/6 | | 2 | | | |
| Taurine/7 | | 2 | | | |
| Beta cyclodextrin/8 | 1 | | | | |
| No ingredient/9 | | | | | 2 |
| (comparison sample) | | | | | |

*+2: strong irritation, +1: medium irritation, 0: a little irritation, −1: little irritation, −2: no irritation

It is observed that the subject ibuprofen troche (5 mg and 10 mg) showed efficacy of pain relief with 8 volunteers, and 10 mg troche showed better result than 5 mg, similar to the results observed with another formulation in Hei.4-26618.

As demonstrated above, a solid dosage formulation containing ibuprofen, such as a lozenge, troche or gum, which further includes the ingredients listed above, can alleviate inflammation or pain in the laryngopharynx by its direct action to the oral mucous membrane and the pharyngeal mucous membrane. The content of ibuprofen per unit is lower than that of the OTC oral preparations and the preparation is safe with no adverse reactions confirmed.

### 11. Test Example

### Effectiveness of Ibuprofen Troche to Relieve Sore Throat, as Well as to Irritant Sensation Masking

(Method) The effectiveness to relieve sore throat was evaluated with volunteers using ibuprofen troches prepared as follows:

1.0 g of ibuprofen was dissolved in 2 ml of EtOH, and 1.0 g of beta-cyclodextrin was kneaded with 0.5 ml of purified water in motor, respectively. The ibuprofen solution was added to the component of beta-cyclodextrin in a motor. The resultant composition was then kneaded well. Afterwards, the composition was dried at room temperature overnight, pulverized by motor, sieved 0.5 mm opening to produce Ibuprofen/beta-cyclodextrin granules.

0.5 g of I-menthol was dissolved in 2 ml of EtOH, and 2.5 g of D-sorbitol was kneaded with 1 ml of purified water in motor, respectively. The I-menthol solution was added to the component of D-sorbitol in a motor. The resultant composition was then kneaded well. Afterwards, the composition was dried at room temperature overnight, pulverized by motor, sieved 0.5 mm opening to produce 1-menthol/D-sorbitol granules.

Then, the ibuprofen/beta-cyclodextrin granules and 1-menthol/D-sorbitol granules were mixed well 0.1 g of Mg-st (magnesium stearate) for 10 seconds. The prepared granules were put into a molder for tabletting (13 mm in diameter, in 1.5-2.0 t pressure for 20 secs.), to obtain 25 troches, each of which weighed 200 mg(40 mg of ibuprofen, 40 mg of beta-cyclodextrin, 20 mg of I-menthol).

(Result)

As shown in Table 2, it was confirmed that the ibuprofen troches worked well to sore throat, and irritant sensation of ibuprofen was reduced by applying formulations.

TABLE 2

| | | | effec- | irri- | |
|---|---|---|---|---|---|
| | | | tiveness | tant | |
| volun- | | gen- | to sore | sensa- | |
| teer | age | der | throat | tion* | remarks |
| 1 | 38 | F | not tested | −2 | Working example 1 was used. |
| 2 | 48 | F | improved | −2 | She got also clearing nasal |
| | | | | | passage. But she preferred |
| | | | | | peppermint flavour to |
| | | | | | l-menthol, because l-menthol |
| | | | | | flavor was too strong for her. |
| 3 | 36 | F | improved | −2 | She also preferred peppermint |
| | | | | | flavor to l-menthol. |
| 4 | 27 | F | improved | 0 | She had no more sore throat |
| | | | | | next day. |
| 5 | 27 | F | not tested | −2 | Peppermint was used instead |
| | | | | | of l-menthol in working |
| | | | | | example 10. |
| 6 | 55 | F | not tested | −1 | Peppermint was used instead |
| | | | | | of l-menthol in working |
| | | | | | example 10. |
| 7 | 56 | M | not tested | −2 | Peppermint was used instead |
| | | | | | of l-menthol in working |
| | | | | | example 10. |
| 8 | 38 | F | not tested | −1 | Peppermint was used instead |
| | | | | | of l-menthol in working |
| | | | | | example 10. |
| 9 | 60 | F | improved | −2 | Peppermint was used instead |
| | | | | | of l-menthol in working |
| | | | | | example 10. The amount of |
| | | | | | main component was different |
| | | | | | from example 10 as follows; |
| | | | | | 20 mg of ibuprofen, 30 mg of |
| | | | | | beta-cyclodextrin, 10 mg of |
| | | | | | peppermint oil |

*+2: strong irritation, +1: medium irritation, 0: a little irritation, −1: little irritation, −2: no irritation

It is evident from the above results and discussion that the subject invention provides important new oral ibuprofen formulations that are organoleptically acceptable and economical to produce. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An organoleptically acceptable solid oral dosage formulation of ibuprofen, said formulation comprising:
ibuprofen; and
a masking component;
wherein said formulation does not include a cyclodextrin in an amount that is two or more times the amount of ibuprofen in said formulation.

2. The formulation according to claim 1, wherein said masking component includes a cooling agent.

3. The formulation according to claim 2, wherein said cooling agent is chosen from I-menthol, dI-menthol, WS-23 (N,2,3-trimethyl-2-isopropyl butamide), WS-3 (cyclohexane carboxamide, N-ethyl-5-methyl-2-(1-methylethyl), peppermint oil and spearmint oil.

4. The formulation according to claim 1, wherein said masking component includes an organic acid.

5. The formulation according to claim 4, wherein said organic acid is chosen from asparaginic acid, citric acid, malic acid, glutamic acid, taurine tartaric acid and succinic acid.

6. The formulation according to claim 1, wherein said masking component includes a cyclodextrin.

7. The formulation according to claim 1, wherein said masking component includes two or more of a cooling agent and an organic acid.

8. The formulation according to claim 7, wherein said masking component includes a cooling agent, an organic acid and a cyclodextrin.

9. The formulation according to claim 1, wherein said solid dosage formulation is a lozenge, troche, tablet, gargle, spray or gum.

10. An organoleptically acceptable solid oral dosage formulation of ibuprofen, said formulation comprising:
(a) ibuprofen; and
(b) a masking component comprising at least one of:
(i) a cooling agent; and
(ii) an organic acid;
wherein said formulation does not include a cyclodextrin in an amount that is two or more times the amount of ibuprofen in said formulation.

11. The formulation according to claim 10, wherein said masking component includes both said cooling agent and said organic acid.

12. The formulation according to claim 10, wherein said formulation includes a cyclodextrin.

13. The formulation according to claim 10, wherein said cooling agent is chosen from I-menthol, dI-menthol, WS-23 and WS-3, peppermint oil and spearmint oil.

14. The formulation according to claim 10, wherein said organic acid is chosen from asparaginic acid, citric acid, malic acid, glutamic acid, taurine tartaric acid and succinic acid.

15. The formulation according to claim 10, wherein said solid dosage formulation is a lozenge, troche, tablet or gum.

16. A method of orally administering ibuprofen to a subject in need thereof, said method comprising;
administering a formulation according to claim 1 to said subject.

17. The method according to claim 16, wherein said method is a method of treating said subject for sore throat or hoarse voice.

18. A method of making a non-irritating oral solid dosage formulation of ibuprofen, said method comprising:
(a) producing an intermediate composition comprising:
ibuprofen; and
a masking component;
wherein said intermediate composition does not include a cyclodextrin in an amount that is two or more times the amount of ibuprofen in said formulation; and
(b) preparing a solid oral dosage formulation from said intermediate composition.

19. The method according to claim 18, wherein said masking component includes at least one of a cooling agent and an organic acid.

20. The method according to claim 18, wherein said solid dosage formulation is a lozenge, troche, tablet or gum.

21. A kit comprising an organoleptically acceptable solid oral dosage formulation of ibuprofen.

22. The kit according to claim 21, wherein said solid dosage formulation is a lozenge, troche, tablet or gum.
